(19) United States
(12) Patent Application Publication
(10) Pub. No.: US 2012/0157790 A1
(43) Pub. Date: Jun. 21, 2012

(54) PHYSICAL EXAMINATION METHOD USING MOBILE TERMINAL, AND GATEWAY AND MOBILE TERMINAL FOR PHYSICAL EXAMINATION

(75) Inventors: Chan Yong PARK, Daejeon (KR); Joon Ho LIM, Daejeon (KR); Soo Jun PARK, Seoul (KR)

(73) Assignee: Electronics and Telecommunications Research Institute, Daejeon (KR)

(21) Appl. No.: 13/310,618

(22) Filed: Dec. 2, 2011

(30) Foreign Application Priority Data
Dec. 21, 2010 (KR) 10-2010-0131990

(51) Int. Cl. A61B 5/00

(52) U.S. Cl. 600/300

(57) ABSTRACT

A physical examination method using a mobile terminal, and a gateway and a mobile terminal for physical examination are provided. The physical examination method using a mobile terminal may include: receiving, at a physical measurement gateway, information on a subject and information on a physical measurement device to be used by the subject through the mobile terminal; and receiving, at the physical measurement gateway, physical information on the subject measured by the physical measurement device, transmitting the physical information to a health management server, receiving physical examination results, obtained based on the transmitted physical information, from the health management server, and providing the physical examination results to the mobile terminal of the subject. Thus, a patient who has limited mobility can easily undergo a physical examination without accessing the physical measurement gateway, thereby constructing a convenient U-health environment.

### CROSS-REFERENCE TO RELATED PATENT APPLICATION

This application claims the benefit of Korean Patent Application No. 10-2010-0131990, filed on Dec. 21, 2010, in the Korean Intellectual Property Office, the disclosure of which is incorporated herein in its entirety by reference.

### BACKGROUND OF THE INVENTION

1. Field of the Invention

The present invention relates to a physical examination method using a mobile terminal, and a physical measurement gateway and a mobile terminal for physical examination and, more particularly, to a method and system which allows a subject to measure his or her personal physical information using a mobile terminal and a physical measurement device during physical examination.

2. Description of the Related Art

At present, patients with diseases related to diabetes and blood pressure can check their blood glucose and blood pressure abnormalities using simple household measurement tools such as blood glucose meters, blood pressure meters, etc.

Extensive research aimed at constructing a home health care network, which allows a subject to perform a simple medical treatment in his or her house by measuring simple medical information using a portable measurement tool such as a blood glucose meter or blood pressure meter and providing the measured medical information to a hospital through a network, has continued to progress.

An environment where the home health care network has been constructed is called “U-Health” which is the concept that health care can be provided anywhere and anytime.

The U-health is a new type of service that protects the health and life of subjects to maintain a healthy life anywhere and anytime using information communication technologies regardless of time and space. The U-health has attracted much attention due to socioeconomic factors such as a change in health care paradigm from diagnosis and treatment to prevention and management, a change in population structure such as aging, and a lack of medical professionals.

The U-health is expected to solve the problems of rapid increase in medical expenses due to aging through daily health care and life sciences and is expected to make up for the lack of medical professionals by providing an efficient medical service.

With the development of very small sensors capable of easily measuring information on health and life, short-range wireless communication technologies such as Bluetooth, Zigbee, etc., and wireless mobile communication technologies, an environment which allows people to access networks anywhere and anytime has been constructed, and thus extensive research related to the U-health has progressed with the development of these communication technologies.

For example, it is possible to implement a system that measures a variety of biometric information regarding the health using a home biometric information measurement device and transmits the information to a service center such that the service center can monitor health status, manage diseases, and handle emergency situations, thereby providing home care.

A mobile health care service is another type of the U-health technology. Unlike the home care system that measures information and provides services in the house, the mobile health care service measures information and provides services even while moving. The mobile health care service requires a wearable or portable sensor module capable of measuring biometric information, and the measured information may be transmitted to a service center through a portable terminal such as a mobile phone. Currently available products for implementing the mobile health care service include, for example, a diabetes phone configured by connecting a blood pressure meter and a mobile phone. The diabetes phone may transmit a blood glucose level measured by the blood pressure meter to the service center through the mobile phone and provide various blood glucose management services such as notification of measurement time to a user through the mobile phone.

Moreover, extensive research and development for wearable biometric signal measurement systems has been actively conducted. The wearable biometric signal measurement system monitors health status anywhere and anytime by mounting a sensor on clothes to measure various information. The wearable biometric signal measurement system has mobility and provides continuous measurement, and thus it can be effectively used when 24-hour monitoring is required.

Global companies such as GE, Philips, etc. leading the medical device market have made large investments in U-health-related businesses. Moreover, IT companies in Korea have been increasingly interested in health information systems and have continuously developed U-health-related products.

In the field of U-health, the standardization has also been carried out to ensure compatibility, extensibility, and interoperability between products.

What is most important in the construction of the U-health environment is that patients can receive medical services anywhere and anytime.

FIG. 1 is a conceptual diagram showing a conventional medical service method using a physical measurement gateway.

Referring to FIG. 1, a subject must log into a physical measurement gateway that can measure health status of the subject and provide the results.

After logging into the physical measurement gateway, the subject measures his or her physical information using a physical measurement device (e.g., a scale, a sphygmomanometer, a blood glucose meter, an oximeter, etc.) and transmits the measured results to the physical measurement gateway.

The physical measurement gateway analyzes the transmitted physical information and displays the analyzed results on a monitor included in the physical measurement gateway such that the subject can receive his or her health-related information through the displayed information.

When a subject logs into the physical measurement gateway by inputting his or her information (e.g., an ID, resident registration number, etc.) to undergo a physical examination, the subject must directly access the physical measurement gateway. However, if the subject is a patient with an advanced disease, it is very difficult for the patient to access the physical measurement gateway without the help of a guide.

The use of a large number of physical measurement gateways to solve the above-described problem may impose an economic burden on the hospital.

### SUMMARY OF THE INVENTION

The present invention has been made in an effort to solve the above-described problems associated with prior art, and a first object of the present invention is to provide a method that allows a subject to undergo a physical examination using a mobile terminal.

A second object of the present invention is to provide a physical measurement gateway for performing a physical examination.

A third object of the present invention is to provide a mobile terminal for performing a physical examination.

According to an aspect of the present invention to achieve the first object of the present invention, there is provided a physical examination method using a mobile terminal, the method comprising the steps of: receiving, at a physical measurement gateway, information on a subject and information on a physical measurement device to be used by the subject through the mobile terminal; and receiving, at the physical measurement gateway, physical information on the subject measured by the physical measurement device, transmitting the physical information to a health management server, receiving physical examination results, obtained based on the transmitted physical information, from the health management server, and providing the physical examination results to the mobile terminal of the subject.

The physical examination method using a mobile terminal may further comprise, when the subject requests additional physical examination information, providing, at the physical measurement gateway, the additional physical examination information to the mobile terminal.

The step of receiving, at the physical measurement gateway, the information on the subject and the information on the physical measurement device to be used by the subject through the mobile terminal may comprise the steps of: receiving, at the physical measurement gateway, registration information of the subject from the mobile terminal; and receiving, at the physical measurement gateway, the information on the physical measurement device to be used by the subject from at least one of the mobile terminal and the physical measurement device and registering the physical measurement device.

The step of receiving, at the physical measurement gateway, the information on the subject and the information on the physical measurement device to be used by the subject through the mobile terminal may further comprise the step of receiving, at the physical measurement gateway, the information on the subject and the information on the physical measurement device to be used by the subject from the mobile terminal.

The step of receiving, at the physical measurement gateway, the physical information on the subject measured by the physical measurement device, transmitting the physical information to the health management server, receiving the physical examination results, obtained based on the transmitted physical information, from the health management server, and providing the physical examination results to the mobile terminal of the subject, may comprise the steps of: transmitting, at the physical measurement gateway, the physical information on the subject, which is measured by the physical measurement device and provided from at least one of the mobile terminal and the physical measurement device, to the health management server; receiving the physical examination results obtained based on the physical information of the subject and provided from the health management server; and providing the physical examination results to the mobile terminal of the subject.

The mobile terminal may use at least one communication network selected from the group consisting of WiFi, Bluetooth, and Zigbee and provide at least one of the information on the subject and the information on the physical measurement device to be used by the subject to the physical measurement gateway.

The step of receiving, at the physical measurement gateway, the information on the subject and the information on the physical measurement device to be used by the subject through the mobile terminal may be performed by an application for physical examination included in the mobile terminal.

According to an aspect of the present invention to achieve the second of the present invention, there is provided a physical measurement gateway for physical examination, the physical measurement gateway comprising: a receiver for receiving information on a subject and information on a physical measurement device to be used by the subject from at least one of a mobile terminal and the physical measurement device; and a transmitter for providing the information on the subject and the information on the physical measurement device to be used by the subject, which are received from the mobile terminal through the receiver, to a physical examination server and providing physical examination results received from the physical examination server to the mobile terminal.

The receiver and the transmitter may use at least one communication network selected from the group consisting of WiFi, Bluetooth, and Zigbee, receive the information on the subject and the information on the physical measurement device to be used by the subject from at least one of the mobile terminal and the physical measurement device, provide the received information on the subject and information on the physical measurement device to be used by the subject to the physical examination server, and provide the physical examination results received from the physical examination server to the mobile terminal.

According to an aspect of the present invention to achieve the third of the present invention, there is provided a mobile terminal for physical examination, the mobile terminal comprising: an input unit for receiving information on a subject and information on a physical measurement device to be used by the subject for the purpose of the physical examination and receiving a request for additional physical examination information from the subject; and a transmitter for transmitting the information on the subject, the information on the physical measurement device to be used by the subject, and the additional physical examination information to a physical measurement gateway in order to connect to a physical examination server that outputs physical examination results obtained based on the information received from the input unit.

The mobile terminal for physical examination may further comprise: a receiver for receiving the physical examination information transmitted from the physical measurement gateway; and an output unit for outputting physical examination results received from the receiver.

The receiver and the transmitter may use at least one communication network selected from the group consisting of WiFi, Bluetooth, and Zigbee, receive the physical examination information transmitted from the physical measurement gateway, and transmit the information on the subject, the information on the physical measurement device to be used by the subject, and the additional physical examination information to the physical measurement gateway.

### BRIEF DESCRIPTION OF THE DRAWINGS

The above and other features and advantages of the present invention will become more apparent by describing in detail exemplary embodiments thereof with reference to the attached drawings in which:

FIG. 1 is a conceptual diagram showing a conventional medical service method using a gateway;

FIG. 2 is a conceptual diagram showing a physical examination system using a mobile terminal in accordance with an exemplary embodiment of the present invention;

FIG. 3 is a conceptual diagram showing a physical examination system which allows a plurality of subjects to undergo a physical examination using a physical measurement gateway in accordance with an exemplary embodiment of the present invention;

FIG. 4 is a flowchart showing a physical examination method using a mobile terminal in accordance with an exemplary embodiment of the present invention;

FIG. 5 is a signal flowchart showing how a user accesses a physical measurement gateway using a mobile terminal;

FIG. 6 is a conceptual diagram showing a physical measurement gateway for physical examination in accordance with an exemplary embodiment of the present invention; and

FIG. 7 is a conceptual diagram showing a mobile terminal for physical examination in accordance with an example embodiment of the present invention.

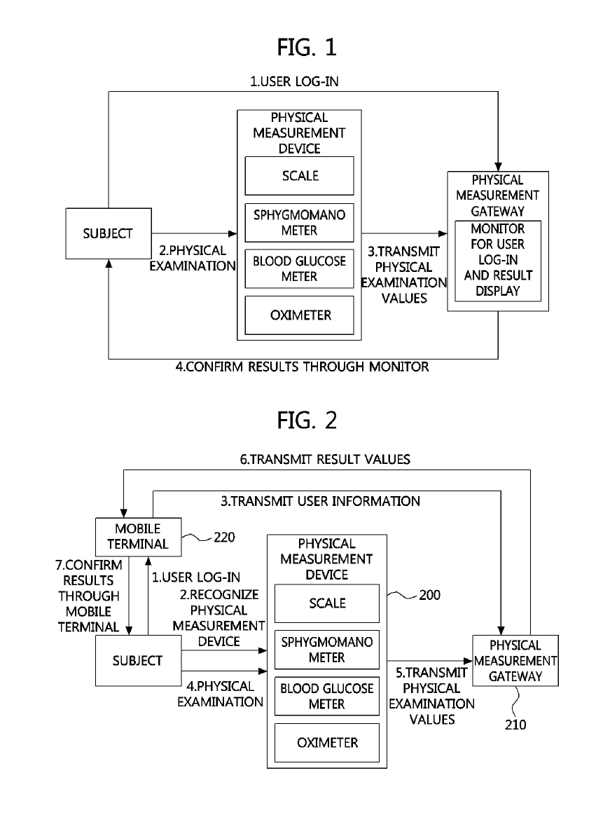

### DETAILED DESCRIPTION OF THE INVENTION

While the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention. Like numbers refer to like elements throughout the description of the figures.

It will be understood that, although the terms first, second, A, B etc. may be used herein to describe various elements, these elements should not be limited by these terms. These terms are only used to distinguish one element from another. For example, a first element could be termed a second element, and similarly, a second element could be termed a first element, without departing from the spirit and scope of the invention. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof.

Unless otherwise defined, all terms, including technical and scientific terms, used herein have the same meaning as commonly understood by one of ordinary skill in the art to which this invention pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, exemplary embodiments of the present invention will be described in detail with reference to the accompanying drawings. Like reference numerals in the drawings denote like elements, and thus repeated descriptions will be omitted.

A mobile terminal used in the following exemplary embodiments of the present invention may be a typical network device such as a smart phone, a notebook, a laptop computer, a cellular phone, etc. that a subject possesses or may be a device specially designed to allow a subject to undergo a physical examination conveniently.

FIG. 2 is a conceptual diagram showing a physical examination system using a mobile terminal in accordance with an exemplary embodiment of the present invention.

Referring to FIG. 2, a physical examination system includes a physical measurement device 200, a physical measurement gateway 210, and a mobile terminal 220.

The physical measurement device 200 measures physical information of a subject and may be a physical information measurement device such as a scale, a sphygmomanometer, a blood glucose meter, or an oximeter.

The above-described scale, sphygmomanometer, blood glucose meter, oximeter are examples of the physical measurement device 200, and various devices capable of obtaining health-related information of a subject may be included in the physical measurement device 200 without departing from the spirit and scope of the invention.

The physical measurement gateway 210 may receive information on the subject transmitted from the mobile terminal 220 and information on the physical measurement device 200 to be used by the subject.

The subject may log into the physical measurement gateway 210 by transmitting his or her information to the physical measurement gateway 210 through the mobile terminal 220.

For example, the subject may be registered in the physical measurement gateway 210 by executing an application included in the mobile terminal 220 and inputting his or her registration information to the application.

After logging into the physical measurement gateway 210, the subject may register the use of the physical measurement device 200 using an identification number, a bar code, a QR code, etc. of the physical measurement device 200 that he or she will use and provide the registration information to the physical measurement gateway 210 through the mobile terminal 220.

Otherwise, if the subject is located around the physical measurement device 200, the use of the physical measurement device 200 may be automatically recognized by the mobile terminal 220 through a local area network provided in both the mobile terminal 220 and the physical measurement device 200, and such information may be provided to the physical measurement gateway 210.

The physical measurement gateway 210 may notify that the physical measurement device 200 has been registered through a display of the mobile terminal 220, and the subject may undergo a physical examination using the physical measurement device 200.

The subject may transmit his or her registration information and the registration information of the physical measurement device to the physical measurement gateway 210 using the mobile terminal such that other subjects cannot use the corresponding physical measurement device, thereby preventing the physical examination record of the corresponding subject from being mixed with those of other subjects.

The mobile terminal 220 may be used to allow a patient to log into the physical measurement gateway 210 with his or her information and to receive the results transmitted from the physical measurement gateway 210.

In an exemplary embodiment of the present invention, the subject may log into the physical measurement gateway 210 using software such as the application included in the mobile terminal 220, the physical information may be provided to the physical measurement gateway 210 from the physical measurement device 200, and the obtained physical examination results may be transmitted to the mobile terminal 220.

Furthermore, the mobile terminal 220 may receive the physical examination results by accessing a website that provides a service for providing physical examination results obtained based on the results measured by the physical measurement device 200.

When a mobile terminal dedicated for the physical examination is used, a subject may log into the physical measurement gateway 210 only by inputting his or her personal information to the mobile terminal in advance and pressing a particular button.

That is, according to an exemplary embodiment of the present invention, various methods may be used to log into the physical measurement gateway using a mobile terminal without departing from the spirit and scope of the invention.

Unlike a conventional physical examination system, according to an exemplary embodiment of the present invention, in which a subject can log into the physical measurement gateway 210 using the mobile terminal 220, the subject can access the physical measurement gateway 210 through a portable mobile terminal and receive the physical examination results through the display of the mobile terminal. As a result, the subject can minimize the distance that subject moves to log into the physical measurement gateway, and the information on a plurality of subjects can be processed at the same time without a plurality of gateways for performing the login operation.

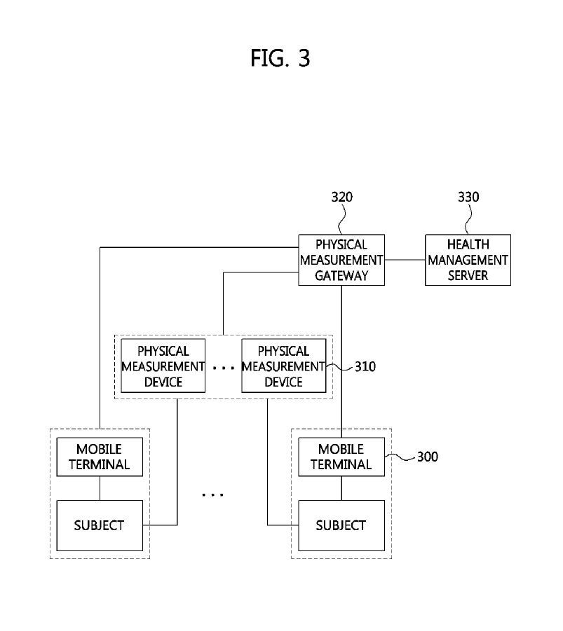

FIG. 3 is a conceptual diagram showing a physical examination system which allows a plurality of subjects to undergo a physical examination using a physical measurement gateway in accordance with an exemplary embodiment of the present invention.

Referring to FIG. 3, a physical examination system in accordance with an exemplary embodiment of the present invention includes a plurality of mobile terminals 300, a physical measurement device 310, a physical measurement gateway 320 and a health management server 330.

Each of a plurality of subjects may have a mobile terminal 300 and log into the physical measurement gateway 320 using the mobile terminal 300.

In the following exemplary embodiment of the present invention, a method that allows a subject to undergo a physical examination using an application included in the mobile terminal will be disclosed. However, the subject may undergo a physical examination through the mobile terminal using any other methods than the above-described method without departing from the spirit and scope of the invention.

In an exemplary embodiment of the present invention, a plurality of the subjects may execute a physical measurement application using their own mobile terminals 300. After logging into the physical measurement gateway 320 through the application, the subject may allow the application to recognize the use of the physical measurement device 310 using an identification number, a bar code, a QR code, etc. of the physical measurement device 310 so as to register the use of the physical measurement device 310 in the physical measurement gateway 320 and may provide the recognized information on the physical measurement device 310 to the physical measurement gateway 320.

Otherwise, if the mobile terminal 300 is located around the physical measurement device 310, the use of the physical measurement device 310 may be automatically recognized by the mobile terminal through a local area network provided in both the mobile terminal 300 and the physical measurement device 310, and such information on the physical measurement device 310 may be provided to the physical measurement gateway 320.

After receiving a registration ACK of the physical measurement device 310 from the physical measurement gateway 320, the mobile terminal 300 may transmit, to the physical measurement gateway 320, the current user login information and an identification information message of the physical measurement device 310 to be used.

After receiving the current user login information and the identification information message of the physical measurement device 310 to be used, the physical measurement gateway 320 may prevent other subjects than the corresponding subject from using the physical measurement device 310.

When the subject undergoes a physical examination through the physical measurement device 310, the measured results may be transmitted from the physical measurement device 310 directly to the physical measurement gateway 320 in a wired or wireless manner or may be transmitted to the physical measurement gateway 320 through the communication network of the mobile terminal 300.

The mobile terminal 300 may request the subject to undergo a physical examination, and then the subject may measure his or her physical information using the physical measurement device 310 in response to the measurement request.

The values measured by the physical measurement device 310 may be transmitted to the physical measurement gateway 320, and the physical measurement gateway 320 may transmit the corresponding values to the health management server 330, thereby managing the personal health record. The measured results may be transmitted to the mobile terminal 300 of the subject and, upon completion of the measurements, the mobile terminal 300 may transmit a physical examination completion message to the physical measurement gateway 320.

When the subject wants to confirm a request for health information such as his or her health information history, a doctor's opinion on health, etc. using the mobile terminal, the mobile terminal may request additional health information from the physical measurement gateway, and the physical measurement gateway may request the additional health information from the health management server.

The health management server may check the login information of the subject and, if the subject is identified, the health management server may transmit the additional health information requested by the subject to the display of the mobile terminal of the subject.

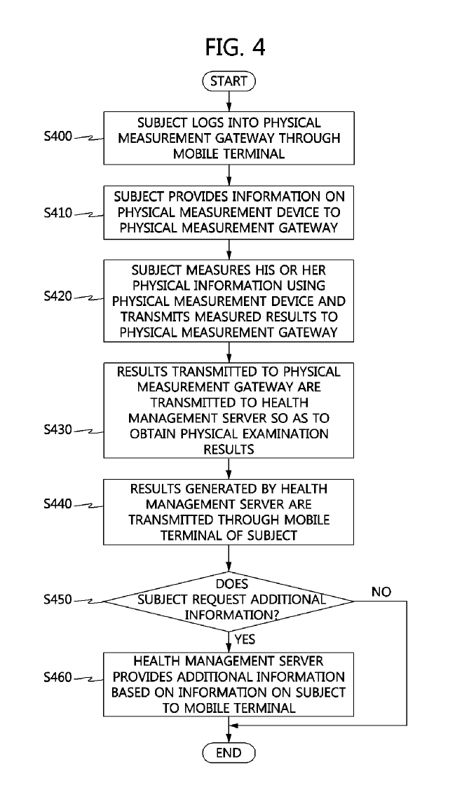

FIG. 4 is a flowchart showing a physical examination method using a mobile terminal in accordance with an exemplary embodiment of the present invention.

Referring to FIG. 4, the subject may log into the physical measurement gateway through the mobile terminal (S400).

The subject may log into the physical measurement gateway by various methods such as using an application dedicated for the physical examination, which is included in the mobile terminal, accessing a web service for providing a physical examination service using the mobile terminal, using a mobile terminal dedicated for the physical examination, etc.

The subject may provide information on the physical measurement device that the he or she will use to the physical measurement gateway (S410).

Various methods may be used to provide the information on the physical measurement device to be used by the subject to the physical measurement gateway.

For example, the use of the physical measurement device may be registered by registering an identification number, a bar code, a QR code, etc. of the physical measurement device of the subject in the mobile terminal. After a response to the request for the registration of the physical measurement device is received from the physical measurement gateway through the mobile terminal, the login information of the current subject and the identification information of the physical measurement device to be used by the subject may be transmitted to the physical measurement gateway.

What's more, the subject may allow the physical measurement gateway to recognize the physical measurement device that he or she will use by receiving the registration information of the physical measurement device through the mobile terminal via a local area network provided in both the mobile terminal and the physical measurement device and by transmitting the information on the physical measurement device to the physical measurement gateway through the communication network of the mobile terminal. Otherwise, the subject may allow the physical measurement gateway to recognize the physical measurement device that he or she will use by providing the information on the corresponding physical measurement device to the physical measurement gateway through a wired/wireless communication network which is provided in each physical measurement device, instead of the communication network of the mobile terminal.

That is, according to an exemplary embodiment of the present invention, the registration of the information on the subject in the physical measurement gateway through the mobile terminal and the registration of the physical measurement device in the physical measurement gateway may be performed independently or dependently. Moreover, this registration process may be performed regardless of chronological order. In other words, while steps S400 and S410 are sequentially described for convenience of description, steps S400 and S410 may be performed independently or dependently regardless of chronological order.

The subject may measure his or her physical information using the physical measurement device storing the identification information of the subject and transmit the measured results to the physical measurement gateway (S420).

After measuring the physical information (such as a blood pressure, a body temperature, a heart rate, etc.) using a physical measurement device such as a blood glucose meter, a body temperature meter, a heart rate meter, etc. the subjects may transmit the results measured by the physical measurement device to the physical measurement gateway through a wired/wireless communication unit provided in the physical measurement device. Otherwise, the subject may receive the physical examination results measured by the physical measurement device through a communication unit installed in the mobile terminal and transmit the measured results to the physical measurement gateway through the communication unit of the mobile terminal.

The results transmitted to the physical measurement gateway may be transmitted to the health management server so as to obtain the physical examination results (S430).

The health management server may store the physical examination information of the previous subjects. Moreover, the health management server may examine the health status of the subject based on the information on the subject transmitted through the physical measurement gateway and the information transmitted from the physical measurement device. The health management server may examine the health status of the subject based on the information on the subject transmitted through the physical measurement gateway and the information measured by the physical measurement device.

When the health management server detects an abnormality, information on the corresponding abnormality may be transmitted to a medical team for the corresponding subject such that the corresponding subject is given a complete medical examination by the medical team.

The results generated by the health management server may be transmitted through the mobile terminal of the subject (S440).

The physical examination information produced by the health management server may be transmitted to the mobile terminal of the subject through the physical measurement gateway such that the subject can check the physical examination information through the display of the mobile terminal.

The subject can see the physical examination results through the mobile terminal, and it can be determined whether the subject requests additional information other than the results (S450).

When the subject wants to compare the current status with the previous record, the subject may additionally request the previous physical examination record stored in the server. When the subject does not request additional information, the physical examination may be completed.

When the subject requests additional information, the health management server may provide the additional information to the mobile terminal of the subject (S460).

When the subject requests his or her previous physical examination record, the server can provide such information. When the subject requests the actual medical team to determine the physical examination results, the server may transmit the request to the medical team and provide the determination result transmitted from the medical team to the subject, thereby providing additional information to the subject in response to the request from the subject.

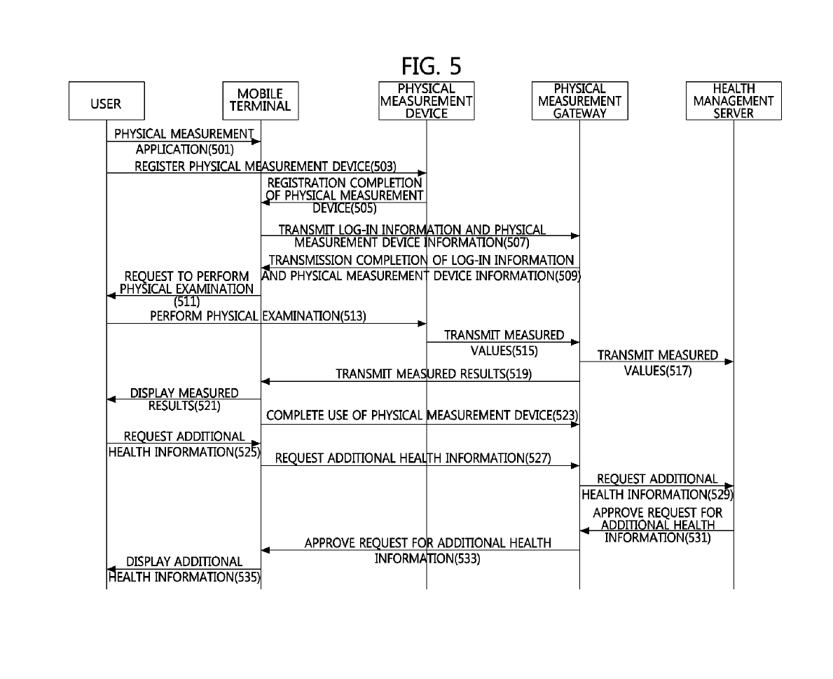

FIG. 5 is a signal flowchart showing how a user accesses the physical measurement gateway using the mobile terminal.

Referring to FIG. 5, the subject may execute a physical measurement application using the mobile terminal (501) and register a physical measurement device that he or she will use using the mobile terminal (503).

After receiving a registration completion message from the physical measurement device (505), the subject may transmit his or her login information and information on the registered physical measurement device to the physical measurement gateway using the mobile terminal (507).

After receiving a transmission completion message for both the login information and the information on the registered physical measurement device from the physical measurement gateway (509), the mobile terminal may request the subject to undergo a physical examination using the physical measurement device (511).

The subject may undergo a physical examination using the physical measurement device (513). Information measured using the physical measurement device may be transmitted to the physical measurement gateway (515) or transmitted to the health management server (517).

The health management server may obtain the physical examination results of the subject based on the transmitted information and transmit the physical examination results to the mobile terminal of the subject through the physical measurement gateway (519). The transmitted information may be provided to the subject through the display of the mobile terminal (521).

The mobile terminal may provide information indicating that the use of the physical measurement device is completed to the physical measurement gateway (523).

When the subject requests additional health information using the mobile terminal (525), the mobile terminal may request the additional health information from the physical measurement gateway (527), and the physical measurement gateway may request the additional health information from the health management server (529).

When the request for the additional health information is approved by the health management server (531), the physical measurement gateway may transmit a message indicating that the request for the additional health information is approved and the additional health information to the mobile terminal (533) and provide the additional health information to the subject through the display of the mobile terminal (535).

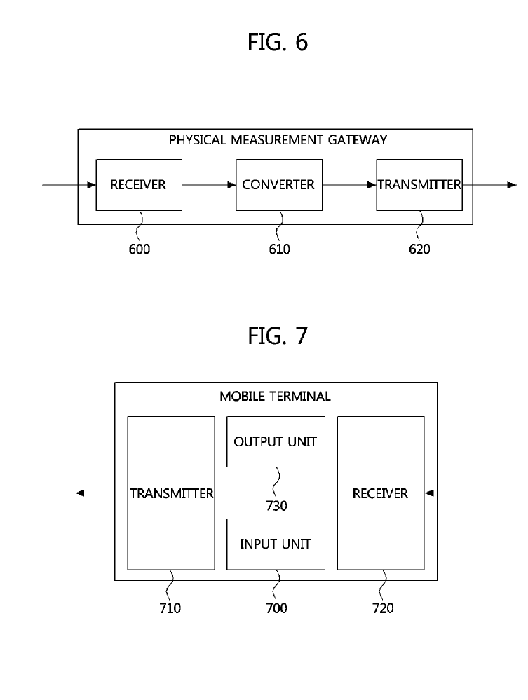

FIG. 6 is a conceptual diagram showing a physical measurement gateway for physical examination in accordance with an exemplary embodiment of the present invention.

Referring to FIG. 6, the physical measurement gateway may comprise a receiver 600, a converter 610 and a transmitter 620.

The receiver 600 may receive information on a subject and information on a physical measurement device, which are transmitted from the mobile terminal, and receive the result information based on the information on the subject and the information on the physical measurement device provided from the health management server.

Moreover, when the subject requests additional physical examination information through the mobile terminal, the receiver 600 may receive the request and receive the additional physical examination information provided by the health management server.

When a network through which the mobile terminal and the physical measurement device transmit the information to the physical measurement gateway is different from a network through which the physical measurement gateway transmits the information to the health management server, or when protocols between the networks are different from each other, the converter 610 may convert the protocols to be appropriate for each network.

When the physical examination results transmitted from the health management server are transmitted to the mobile terminal of the subject, or when the subject requests additional physical examination information, the transmitter 620 may transmit the additional physical examination information received from the health management server to the subject.

Moreover, the transmitter 620 may transmit the information on the subject, the information on the physical measurement device, and the additional physical examination information, which are transmitted through the receiver 600, to the health management server.

The receiver 600 and the transmitter 620 may be implemented by way of a local area network such as Bluetooth, WiFi, Zigbee, etc. to transmit and receive the information between the physical measurement gateway and the mobile terminal.

While the transmitter 620 and the receiver 600 have been described as individual components for convenience of description, the transmitter 620 and the receiver 600 may be integrated into a single communication unit.

FIG. 7 is a conceptual diagram showing a mobile terminal for physical examination in accordance with an example embodiment of the present invention.

Referring to FIG. 7, the mobile terminal for physical examination may comprise an input unit 700, a transmitter 710, a receiver 720 and an output unit 730.

The input unit 700 may receive information on a subject and information on a physical measurement device to be used by the subject for the purpose of the physical examination and receive a request for additional physical examination information from the subject.

The transmitter 710 may transmit the information provided by the input unit 700 to a physical measurement gateway.

That is, in order to connect to a physical examination server that outputs the physical examination results obtained based on the information received from the input unit 700, the transmitter 710 may transmit the information on the subject, the information on the physical measurement device to be used by the subject, and the additional physical examination information to the physical measurement gateway.

The receiver 720 may receive the physical examination information transmitted from the physical measurement gateway.

The output unit 730 may output the information received through the receiver 720 to the mobile terminal.

While the receiver 720 and the output unit 730 have been described as being included in the mobile terminal for convenience of description, the receiver 720 and the output unit 730 may be implemented as a separate device from the mobile terminal including the input unit 700 and the transmitter 710.

For example, the subject may undergo a physical examination using a simplified mobile terminal configured only to input information and to transmit the input information without the output unit 730 and the transmitter 710 and may receive the physical examination results through a mobile terminal, which includes the output unit 730 and the transmitter 710, such as a notebook or smart phone that he or she possesses.

The receiver 720 and the transmitter 710 may be implemented by way of a local area network such as Bluetooth, WiFi, Zigbee, etc. to transmit and receive the information between the physical measurement gateway and the mobile terminal.

While the transmitter 720 and the receiver 720 have been described as individual components for convenience of description, the transmitter 710 and the receiver 720 may be integrated into a single communication unit.

As described above, according to the physical examination method and system using a mobile terminal in accordance with the exemplary embodiments of the present invention, when a subject accesses a physical measurement gateway using a mobile terminal, the physical examination results obtained by the physical measurement gateway can be provided to the subject through the mobile terminal of the subject.

Therefore, a subject who has limited mobility can easily log into the physical measurement gateway using the mobile terminal and receive the physical examination results through the display of the mobile terminal without accessing the physical measurement gateway.

While the invention has been particularly shown and described with reference to exemplary embodiments thereof, it will be understood by those of ordinary skill in the art that various changes in form and details may be made therein without departing from the spirit and scope of the invention as defined by the following claims.

What is claimed is:

1. A physical examination method using a mobile terminal, the method comprising:
receiving, at a physical measurement gateway, information on a subject and information on a physical measurement device to be used by the subject through the mobile terminal; and
receiving, at the physical measurement gateway, physical information on the subject measured by the physical measurement device, transmitting the physical information to a health management server, receiving physical examination results, obtained based on the transmitted physical information, from the health management server, and providing the physical examination results to the mobile terminal of the subject.

2. The method of claim 1, further comprising, when the subject requests additional physical examination information, providing, at the physical measurement gateway, the additional physical examination information to the mobile terminal.

3. The method of claim 1, wherein the receiving, at the physical measurement gateway, the information on the subject and the information on the physical measurement device to be used by the subject through the mobile terminal comprises:
receiving, at the physical measurement gateway, registration information of the subject from the mobile terminal; and
receiving, at the physical measurement gateway, the information on the physical measurement device to be used by the subject from at least one of the mobile terminal and the physical measurement device and registering the physical measurement device.

4. The method of claim 3, wherein the receiving, at the physical measurement gateway, the information on the subject and the information on the physical measurement device to be used by the subject through the mobile terminal further comprises receiving, at the physical measurement gateway, the information on the subject and the information on the physical measurement device to be used by the subject from the mobile terminal.

5. The method of claim 1, wherein the receiving, at the physical measurement gateway, the physical information on the subject measured by the physical measurement device, transmitting the physical information to the health management server, receiving the physical examination results, obtained based on the transmitted physical information, from the health management server, and providing the physical examination results to the mobile terminal of the subject, comprises:
transmitting, at the physical measurement gateway, the physical information on the subject, which is measured by the physical measurement device and provided from at least one of the mobile terminal and the physical measurement device, to the health management server;
receiving the physical examination results obtained based on the physical information of the subject and provided from the health management server; and
providing the physical examination results to the mobile terminal of the subject.

6. The method of claim 1, wherein the mobile terminal uses at least one communication network selected from the group consisting of WiFi, Bluetooth, and Zigbee and provides at least one of the information on the subject and the information on the physical measurement device to be used by the subject to the physical measurement gateway.

7. The method of claim 1, wherein the receiving, at the physical measurement gateway, the information on the subject and the information on the physical measurement device to be used by the subject through the mobile terminal is performed by an application for physical examination included in the mobile terminal.

8. A physical measurement gateway for physical examination, the physical measurement gateway comprising:
a receiver for receiving information on a subject and information on a physical measurement device to be used by the subject from at least one of a mobile terminal and the physical measurement device; and
a transmitter for providing the information on the subject and the information on the physical measurement device to be used by the subject, which are received from the mobile terminal through the receiver, to a physical examination server and providing physical examination results received from the physical examination server to the mobile terminal.

9. The physical measurement gateway of claim 8, wherein the receiver and the transmitter use at least one communication network selected from the group consisting of WiFi, Bluetooth, and Zigbee, receive the information on the subject and the information on the physical measurement device to be used by the subject from at least one of the mobile terminal and the physical measurement device, provide the received information on the subject and information on the physical measurement device to be used by the subject to the physical examination server, and provide the physical examination results received from the physical examination server to the mobile terminal.

10. A mobile terminal for physical examination, the mobile terminal comprising:
an input unit for receiving information on a subject and information on a physical measurement device to be used by the subject for the purpose of the physical examination and receiving a request for additional physical examination information from the subject; and
a transmitter for transmitting the information on the subject, the information on the physical measurement device to be used by the subject, and the additional physical examination information to a physical measurement gateway in order to connect to a physical examination server that outputs physical examination results obtained based on the information received from the input unit.

11. The mobile terminal of claim 10, further comprising:
a receiver for receiving the physical examination information transmitted from the physical measurement gateway; and
an output unit for outputting physical examination results received from the receiver.

12. The mobile terminal of claim 11, wherein the receiver and the transmitter use at least one communication network selected from the group consisting of WiFi, Bluetooth, and Zigbee, receive the physical examination information transmitted from the physical measurement gateway, and transmit the information on the subject, the information on the physical measurement device to be used by the subject, and the additional physical examination information to the physical measurement gateway.
